(19) United States
(12) Patent Application Publication
(10) Pub. No.: US 2016/0211045 A1
(43) Pub. Date: Jul. 21, 2016

(54) X-RAY IMAGING APPARATUS AND METHOD OF CONTROLLING THE SAME

(71) Applicant: SAMSUNG ELECTRONICS CO., LTD., Suwon-si (KR)

(72) Inventors: Sung W. JEON, Suwon-si (KR); Young Hun SUNG, Hwaseong-si (KR)

(73) Assignee: SAMSUNG ELECTRONICS CO., LTD., Suwon-si (KR)

(21) Appl. No.: 14/877,220

(22) Filed: Oct. 7, 2015

(30) Foreign Application Priority Data
Jan. 20, 2015 (KR) 10-2015-0009109

(51) Int. Cl. G21K 1/10; A61B 6/00; G01N 23/04

(52) U.S. Cl. CPC G21K 1/10; G01N 23/04; A61B 6/504

(57) ABSTRACT

An X-ray imaging apparatus includes an X-ray source configured to radiate X-rays to an object; an X-ray detector configured to detect X-rays radiated by the X-ray source; and a disk rotatably provided between the X-ray source and the X-ray detector, wherein region of interest (ROI) filters are configured to filter X-rays radiated by the X-ray source are mounted on the disk, the ROI filters having openings of different sizes.

### CROSS-REFERENCE TO RELATED APPLICATION

This application claims priority from Korean Patent Application No. 10-2015-0009109, filed on Jan. 20, 2015, in the Korean Intellectual Property Office, the disclosure of which is incorporated herein in its entirety by reference.

### BACKGROUND

1. Field

Apparatuses and methods consistent with exemplary embodiments relate to an X-ray imaging apparatus that radiates X-rays to an object and images an inside thereof, and a method of controlling the same.

2. Description of the Related Art

An X-ray imaging apparatus is an apparatus capable of obtaining an internal image of an object by radiating X-rays to the object and using the X-rays passing through the object. Since permeability of X-rays differs depending on properties of a substance forming the object, it is possible to image an internal structure of the object by detecting an intensity or a strength of X-rays passing through the object.

In order to ensure safety in using the X-ray imaging apparatus, reducing a dose of X-rays incident on the object is desired.

### SUMMARY

One or more exemplary embodiments provide an X-ray imaging apparatus capable of implementing low dose X-ray imaging and minimizing a loss of a field of view (FOV) of an X-ray image by providing a smaller dose of X-rays than that on a region of interest (ROI) to be incident on a non-ROI using an ROI filter, and a method of controlling the same.

One or more exemplary embodiments also provide an X-ray imaging apparatus that can be applied to a field of X-ray videos by synchronizing movement of an ROI with movement of an ROI filter and a method of controlling the same.

According to an aspect of an exemplary embodiment, provided is an X-ray imaging apparatus including: an X-ray source configured to radiate X-rays to an object; an X-ray detector configured to detect X-rays radiated by the X-ray source; and a disk rotatably provided between the X-ray source and the X-ray detector, wherein region of interest (ROI) filters are configured to filter X-rays radiated by the X-ray source are mounted on the disk, the ROI filters having openings of different sizes.

The ROI filters may be configured to filter the X-rays such that the filtered X-rays have a smaller dose than the X-rays passing through the openings of the ROI filters

The X-ray imaging apparatus may further include a controller configured to control such that the disk is moved or rotated according to movement of a region of interest (ROI) of the object or a size of the ROI.

The controller may be configured to control such that the disk rotates about an axis that is parallel to a line connecting the X-ray source and the X-ray detector according to the size of the ROI.

The controller may be configured to control such that the disk moves on a plane that is perpendicular to a line connecting the X-ray source and the X-ray detector according to the movement of the ROI.

A number of the ROI filters having the openings may be less than a number of openings formed on the disk.

The ROI filters may be detachably mounted on the disk.

A thickness of an ROI filter in an area adjacent to an opening may be smaller than a thickness of the ROI filter in an area adjacent to an outer diameter of the ROI filter.

The controller may be configured to control to rotate the disk such that the X-rays are filtered by an ROI filter having an opening of which a size corresponds to the size of the ROI.

An ROI filter may have a circular shape.

An opening may have a circular shape.

An opening may have a polygonal shape.

The X-ray detector may be configured to detect the X-rays radiated to the object and obtains a plurality of frame images by using the detected X-rays.

The X-ray imaging apparatus may further include an image processor configured to obtain information on the ROI from at least one of the plurality of frame images and transmit the information to a controller, wherein the controller controls the disk based on the information.

The image processor may be configured to detect an object of interest from the frame image and set the ROI based on at least one of a position, a size, and a movement of the object of interest.

According to an aspect of an exemplary embodiment, provided is a method of controlling an X-ray imaging apparatus including a disk on which a plurality of ROI filters having openings of different sizes are mounted, the method including: radiating, by using an X-ray source, X-rays to an object; detecting, by using an X-ray detector, the radiated X-rays and obtaining information on a region of interest (ROI) of the object; and controlling the disk positioned between the X-ray source and the X-ray detector to rotate corresponding to an area of the ROI.

The controlling may include controlling the disk to move on a plane that is parallel with a plane on which the ROI is positioned.

The controlling may further include controlling the disk to rotate about an axis that is parallel to a line connecting the X-ray source and the X-ray detector and move on a plane that is perpendicular to the line connecting the X-ray source and the X-ray detector.

According to an aspect of an exemplary embodiment, provided is an X-ray imaging apparatus, including: a plurality of region of interest (ROI) filters having openings of different sizes and configured to filter X-rays generated from an X-ray source, the filtered X-rays being radiated to an object; a disk on which the plurality of ROI filters are mounted; and a controller configured to control such that the disk is rotated or moved corresponding to a region of interest (ROI) of the object.

The controller may be configured to move the disk on a plane that is parallel with a plane on which the ROI is positioned.

### BRIEF DESCRIPTION OF THE DRAWINGS

The above and/or other aspects will become more apparent by describing certain exemplary embodiments with reference to the accompanying drawings in which:

FIG. 1 is a control block diagram illustrating an X-ray imaging apparatus according to an exemplary embodiment;

FIG. 2 is a cross sectional view of an internal structure of an X-ray tube included in an X-ray imaging apparatus according to an exemplary embodiment;

FIG. 3 is a diagram illustrating an example of a region of interest (ROI) when a stent is inserted into a blood vessel;

FIG. 4A is a side cross-sectional view of an ROI filter according to an exemplary embodiment;

FIGS. 4B and 4C are plan views of an ROI filter according to an exemplary embodiment;

FIGS. 5A and 5B are diagrams schematically illustrating doses of X-rays incident on an ROI and a non-ROI according to an exemplary embodiment;

FIG. 6A is a diagram illustrating movement of an ROI according to movement of an object of interest according to an exemplary embodiment;

FIG. 6B is a diagram schematically illustrating an operation of tracking a moving ROI according to an exemplary embodiment;

FIG. 6C is a diagram illustrating movement of an ROI filter according to movement of an ROI according to an exemplary embodiment;

FIGS. 7A and 7B are diagrams illustrating a state in which a disk including ROI filters rotates according to a size of an ROI according to an exemplary embodiment;

FIG. 8 is a side cross-sectional view of a disk including an ROI filter according to an exemplary embodiment;

FIGS. 9A, 9B, and 9C are side cross-sectional views of an X-ray imaging apparatus including a disk having an ROI filter according to an exemplary embodiment;

FIGS. 10A, 10B, and 10C are diagrams schematically illustrating doses of X-rays incident on an ROI and a non-ROI according to an exemplary embodiment;

FIG. 11 is a diagram illustrating a disk including ROI filters according to another exemplary embodiment; and

FIG. 12 is a diagram schematically illustrating doses of X-rays incident on an ROI and a non-ROI according to another exemplary embodiment.

### DETAILED DESCRIPTION

Certain exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

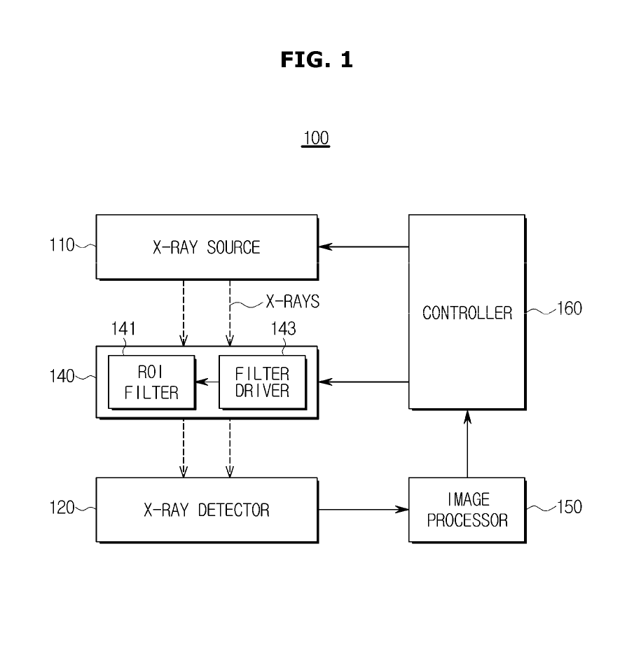

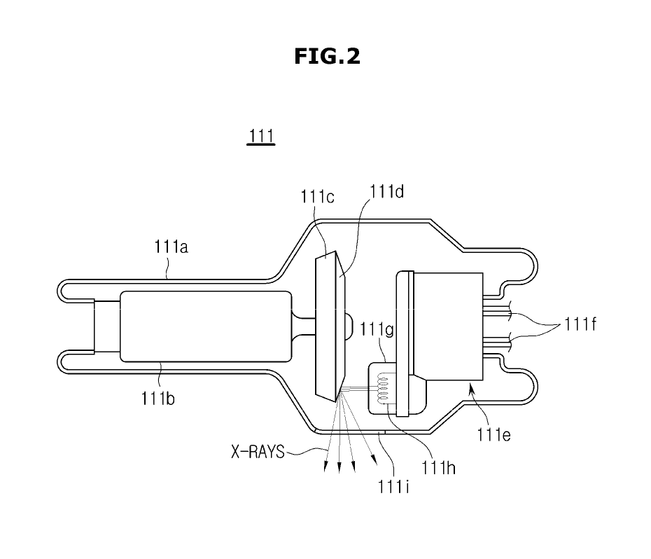

FIG. 1 is a control block diagram illustrating an X-ray imaging apparatus according to an exemplary embodiment. FIG. 2 is a cross sectional view of an internal structure of an X-ray tube included in an X-ray imaging apparatus according to an exemplary embodiment.

As illustrated in FIG. 1, an X-ray imaging apparatus 100 includes an X-ray source 110 configured to generate X-rays and radiate the X-rays, an X-ray detector 120 configured to detect the radiated X-rays and obtain a frame image, a filter 140 configured to filter X-rays generated from the X-ray source 110, an image processor 150 configured to detect a region of interest (ROI) from the obtained frame image, and a controller 160 configured to control the filter 140.

As illustrated in FIG. 2, the X-ray source 110 may include an X-ray tube 111 configured to generate X-rays. An anode 111b and a cathode 111e are provided inside a glass tube 111a of the X-ray tube 111. An inside of the glass tube 111a remains in a high vacuum state, and a filament 111h of the cathode 111e is heated to generate thermoelectrons. When a current is applied to an electrical conductor 111f connected to the filament 111h, the filament 111h may be heated.

The cathode 111e includes the filament 111h and a focusing electrode 111g configured to focus electrons. The focusing electrode 111g is also referred to as a focusing cup.

Also, when a high voltage is applied between the anode 111b and the cathode 111e, thermoelectrons are accelerated and collide with a target material 111d of the anode 111b to generate X-rays. High-resistance materials such as Cr, Fe, Co, Ni, W, and Mo may be used as the target material 111d of the anode 111b. The generated X-rays are radiated to the outside through a window 111i. For example, a beryllium (Be) thin film may be used as a material of the window 111i.

The voltage applied between the anode 111b and the cathode 111e is referred to as a tube voltage, and a level thereof may be indicated as a peak kilovoltage (kvp). As the tube voltage increases, speeds of thermoelectrons increase. As a result, energy (or photon energy) generated by the X-rays colliding with the target material 111d increases. Also, a filter is disposed in a direction in which X-rays are radiated to control energy of X-rays. A filter configured to filter X-rays of a specific wavelength band may be positioned in front of or behind the window 111i to filter X-rays of a specific energy band. For example, when a filter including aluminum or copper is disposed, X-rays of a low energy band are filtered and energy of radiated X-rays increases.

A current flowing in the X-ray tube 111 is referred to as a tube current and may be indicated as an average value (mA). The product of the tube current and the X-ray exposure time is referred to as a tube current-exposure time product (mAs). As the tube current increases, a dose of X-rays (or the number of X-ray photons) increases. Therefore, energy of X-rays may be controlled by the tube voltage. A dose of X-rays may be controlled by the tube current and the X-ray exposure time, that is, the tube current-exposure time product (mAs).

The X-ray imaging apparatus 100 may apply fluoroscopy to generate an X-ray video, and may be applied to X-ray diagnostic fields such as angiography or various procedure fields using the same. In this case, the X-ray video may be generated and displayed in real time.

The X-ray imaging apparatus 100 may continuously perform X-ray imaging to generate the X-ray video. As a method of continuously performing X-ray imaging, a continuous exposure method and a pulse exposure method are provided. When the continuous exposure method is applied, a low tube current is continuously supplied to the X-ray tube 111 and X-rays are continuously generated. When the pulse exposure method is applied, X-rays are generated according to a continuous short pulse. Therefore, when the pulse exposure method is applied, a dose of X-rays and motion blurring may decrease. The X-ray imaging apparatus 100 may use both methods. However, for convenience of description, an application of the pulse exposure method will be described in the following exemplary embodiment.

The X-ray source 110 may radiate X-rays to a region of an object (or an object region) a plurality of times at a predetermined time interval or an arbitrary time interval. Here, the predetermined time interval or the arbitrary time interval may be determined according to a pulse rate or a frame rate. The pulse rate may be determined according to the frame rate or vice versa. The frame rate may be set to 30 frames per second (30 fps), 15 frames per second (15 fps), 7.5 frames per second (7.5 fps), or the like. For example, when the frame rate is set to 15 fps, the pulse rate is set to 15 pps, and X-rays are generated 15 times per second, or when the pulse rate is set to 7.5 pps, X-rays may be generated 7.5 times per second.

The object is an imaging target whose inside is represented as an X-ray image. An object region is a predetermined area including the object and refers to an area to be imaged as an X-ray image. Therefore, the object region may correspond to a field of view (FOV) of the X-ray imaging apparatus 100 or include a field of view of the X-ray imaging apparatus 100.

The object region may include at least one of an ROI and a non-ROI. A region other than the ROI in the object region is designated as the non-ROI. The ROI and the non-ROI will be described later in detail.

The X-ray detector 120 detects X-rays and obtains a plurality of frame images of the object region. The frame images refer to a plurality of X-ray images obtained according to the frame rate of the X-ray imaging apparatus 100. The X-ray detector 120 may include a two dimensional (2D) array structure including a plurality of pixels. When the detected X-rays are converted into an electrical signal for each pixel, one X-ray image of the object region is obtained.

The X-ray detector 120 may use any of various structures in which X-rays are detected and converted into an electrical signal. As an example, any of a direct method in which X-rays are directly converted into an electrical signal using a photoconductor such as a-Se, and an indirect method in which X-rays are converted into visible light using a scintillator such as Csl and the visible light is converted into an electrical signal may be applied.

The filter 140 includes an ROI filter 141 including a material absorbing X-rays and a filter driver 143 configured to move the ROI filter 141. The filter driver 143 may include a mechanical structure such as a motor configured to generate power and a gear configured to deliver the generated power to the ROI filter 141.

The ROI filter 141 may filter X-rays generated from the X-ray source 110 such that a smaller X-ray dose than that on the ROI is incident on the non-ROI. In this manner, the X-ray dose may be reduced. A higher X-ray dose is applied to the ROI, which is of more interest to a user than the non-ROI, and a smaller X-ray dose is applied to the non-ROI than the ROI, through X-ray filtering. Since X-rays are also incident on the non-ROI, the field of view (FOV) of the X-ray imaging apparatus 100 may not be affected. A further detailed structure and operations of the filter 140 will be described later.

As described above, the X-ray imaging apparatus 100 may continuously perform X-ray imaging and obtain an X-ray video of the object region. Frame images obtained by the X-ray detector 120 are input to the image processor 150. The image processor 150 may analyze the input frame images and obtain information on the ROI. When the information on the ROI is delivered to the controller 160, the controller 160 controls the filter 140 such that a smaller X-ray dose than that on the ROI may be incident on the non-ROI.

Operations of the image processor 150 which obtains information on the ROI will be described below in detail.

The image processor 150 may detect an object of interest from the frame image of the object region. To detect the object of interest, features of the object of interest may be stored in advance, and an object corresponding to the pre-stored features may be detected from the frame image of the object region. For example, a feature of the object of interest such as, for example, a shape, an X-ray absorption characteristic and a movement characteristic of the object of interest that is detectable from the X-ray image may be stored in advance. Here, the movement characteristic of the object of interest may include information on movement of the object of interest, and the information on movement may include a movement direction, a movement speed and a position change.

The object of interest is an object that is continuously watched by a user during X-ray imaging and may be an instrument used in the object or a procedure area. For example, if the X-ray imaging apparatus 100 is used for angiography, when an instrument such as a guide wire, a catheter, a needle, a balloon, or a stent is inserted into a blood vessel, these instruments need to be carefully observed. Therefore, the instrument may be set as the object of interest and information on features thereof may be stored in advance.

Also, when the procedure area is set as the object of interest, an area such as a stenosis, an aneurysm or a cancerous region may be the object of interest.

When the object of interest is detected, the image processor 150 may set a predetermined area including the detected object of interest as the ROI. Therefore, a position and a size of the ROI may be determined in consideration of a position and a size of the object of interest or a movement characteristic of the object of interest.

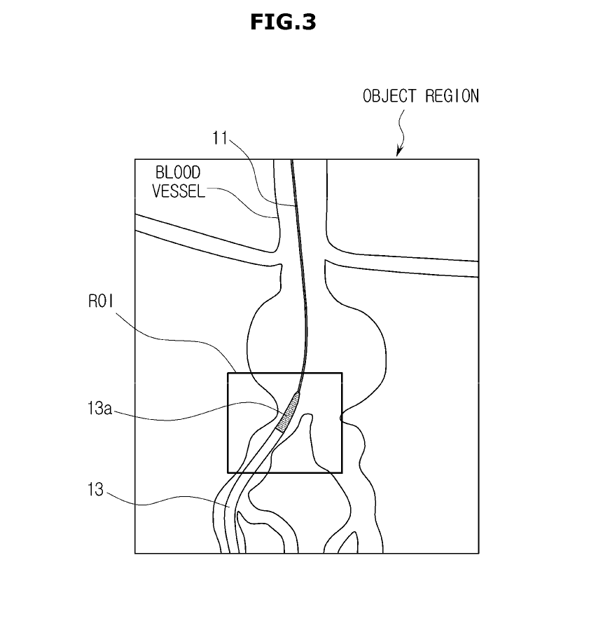

FIG. 3 is a diagram illustrating an example of a region of interest (ROI) when a stent is inserted into a blood vessel. An example of setting an ROI will be described below with reference to FIG. 3.

A stent 13a is inserted into a blood vessel to prevent a blood vessel from being blocked and has a mesh shape. The stent 13a is folded and installed at an end of a stent device 13 having a long tube shape, is introduced into the blood vessel, and is spread at a desired position in a mesh shape.

As illustrated in FIG. 3, to insert the stent device 13 into the blood vessel of the object region, a guide wire 11 is inserted first. The stent device 13 is inserted into the blood vessel along the guide wire 11. While the stent device 13 is inserted, the stent device 13, and specifically, a tip of the stent 13a may be the object of interest, and a predetermined area including the stent 13a may be the ROI.

While the guide wire 11 is inserted, a guide wire 11 or a tip of the guide wire 11 may be the object of interest. Although not illustrated, while the catheter is inserted to inject a contrast agent into the blood vessel, the catheter or a tip of the catheter may be the object of interest.

The image processor 150 may use information input from the outside to detect the object of interest. For example, when information on a kind of the instrument, a kind of the procedure, and a procedure area, and information on injection of the contrast agent and the like are input, the object of interest may be detected from the frame image based on the input information.

As an example, when information that a procedure to be performed is an aortic stenting procedure and an instrument to be inserted is a stent device is input, the image processor 150 uses information on the pre-stored features of the stent and detects a stent in the aorta from the frame image of the object region.

The image processor 150 may determine the movement characteristic of the object of interest while tracking the detected object of interest. Detecting and tracking of the object of interest and obtaining information on the ROI including the object of interest may be performed in real time according to the frame rate of the frame images input to the image processor 150. Here, obtaining the information on the ROI includes detecting and tracking the object of interest and setting the ROI based on a result of the detecting and tracking of the object of interest.

The movement characteristic of the object of interest includes information on a movement size, a movement direction and the like of the object of interest. The movement of the object of interest includes a motion of the object of interest. The movement size may include a speed, but the movement of the object of interest may have no constant pattern. Therefore, the movement size may include various pieces of information indicating a degree of the movement in addition to the speed.

The ROI is a predetermined area including the object of interest and is defined by the object of interest. According to the movement characteristic of the object of interest, the movement characteristic of the ROI may be determined.

Also, the information on the ROI obtained by the image processor 150, and specifically, information on the position, the size or the movement characteristic of the ROI, is transmitted to the controller 160 and used to control the filter 140.

The image processor 150 may obtain information on image characteristics such as noise and contrast represented in the frame image. These characteristics may be transmitted to the controller 160, used to control X-ray imaging conditions, or used to determine a difference of X-ray doses incident on the ROI and the non-ROI.

As described above, when the image processor 150 analyzes the frame image of the object region and obtains information on the ROI, the ROI filter 141 whose movement is controlled by the controller 160 filters X-rays incident on the non-ROI such that low dose X-rays are incident on the non-ROI.

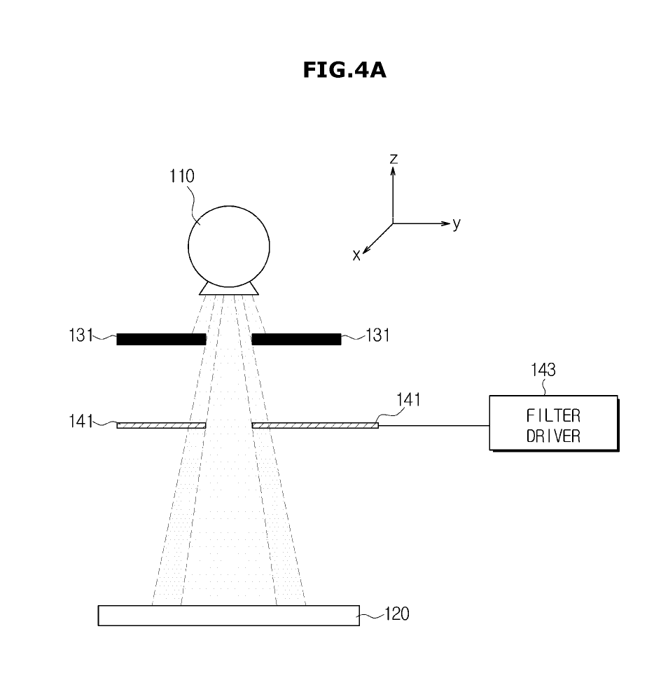

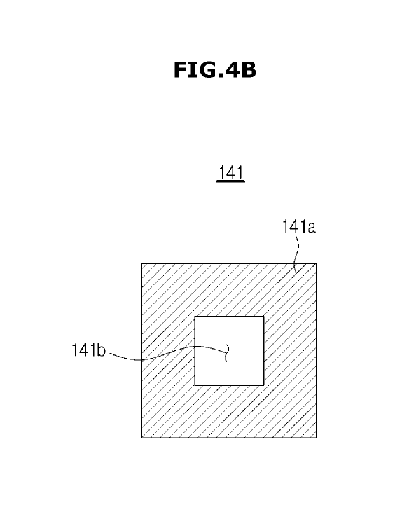

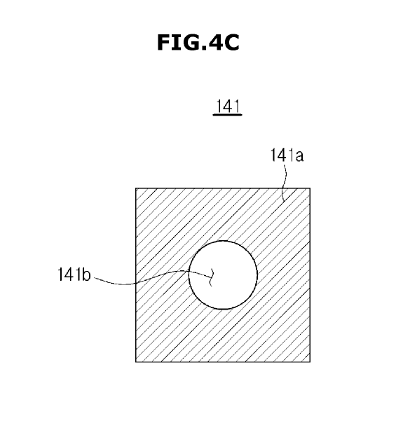

FIG. 4A is a side cross-sectional view of an ROI filter according to an exemplary embodiment. FIGS. 4B and 4C are plan views of an ROI filter according to exemplary embodiments.

As illustrated in FIG. 4A, a collimator 131 may be disposed in front of the X-ray source 110, corresponding to a direction in which X-rays are radiated. The collimator 131 includes material that absorbs or blocks X-rays such as lead or tungsten, controls a range of the field of view (FOV) corresponding to an X-ray irradiation area of the X-ray source 110, and reduces X-ray scattering.

The ROI filter 141 is positioned between the collimator 131 and the X-ray detector 120, and may filter X-rays incident on the non-ROI among X-rays generated from the X-ray source 110. The ROI filter 141 may include a material that attenuates X-rays. Various materials having an X-ray attenuating characteristic such as aluminum (Al), copper (Cu), zirconium (Zr), molybdenum (Mo), silver (Ag), iridium (Ir), iron (Fe), tin (Sn), platinum (Pt), gold (Au), or tantalum (Ta) or mixtures thereof may be used to form the ROI filter 141. The material having such an X-ray attenuating characteristic may be referred to as a filtration material.

The ROI filter 141 may move in a three dimensional (3D) space defined by x, y, and z axes and be positioned at a position corresponding to the non-ROI. Here, the z axis corresponds to a vertical line connecting the X-ray source 110 and the X-ray detector 120, and the x axis and the y axis are perpendicular to the z axis.

For example, the ROI filter 141 may move on an x-y plane or along the z axis. By moving the ROI filter 141 on the x-y plane, positions of the ROI filter 141 and the non-ROI may be matched with each other. By moving the ROI filter 141 along the z axis or in a z axis direction, sizes of the ROI filter 141 and the ROI may be matched with each other.

The ROI may be surrounded by the non-ROI and thus, as illustrated in FIGS. 4B and 4C, an opening 141b may be formed at a center of the ROI filter 141. The opening 141b may have various shapes such as a circle or a polygon. The vicinity of the opening 141b is surrounded by a filtration material 141a.

As an example, a rectangular opening 141b may be provided as illustrated in FIG. 4B or a circular opening 141b may be provided as illustrated in FIG. 4C. The shape of the opening 141b of the ROI filter 141 is not limited thereto, but the ROI filter 141 may have various shapes according to a characteristic of the ROI, a geometric relation between the ROI and the non-ROI and the like.

When X-rays pass through a certain material, a dose of X-rays decreases or X-rays are filtered while passing through the certain material. On the other hand, when X-rays pass through an opening, a dose of X-rays does not decrease or X-rays are not filtered and the X-rays maintain the same property.

Among X-rays generated from the X-ray source 110, X-rays incident on the filtration material 141a have a dose that is reduced while penetrating the filtration material 141a, and X-rays incident on the opening 141b have a dose that is maintained while passing through the opening 141b. Therefore, when the ROI filter 141, and more accurately, the filtration material of the ROI filter 141, is positioned at a position corresponding to the non-ROI within the object region, a smaller dose of X-rays than that on the ROI may be incident on the non-ROI. For example, an amount of X-rays incident on the non-ROI may be ⅕, 1/10 or 1/20 or less of an amount of X-rays incident on the ROI.

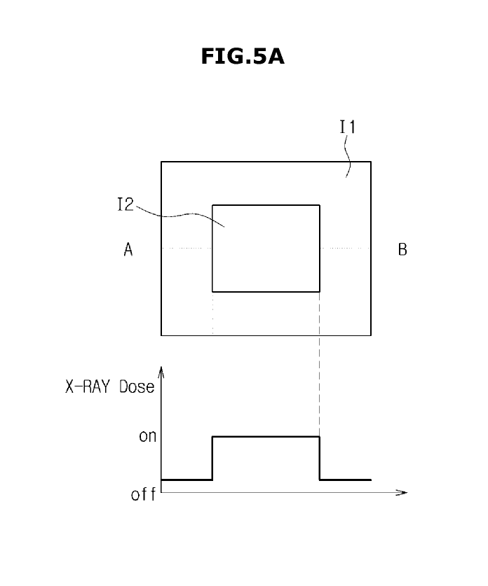

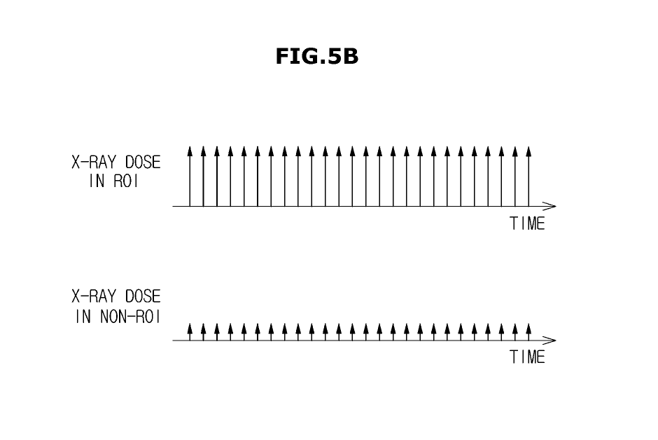

FIGS. 5A and 5B are diagrams schematically illustrating doses of X-rays incident on an ROI and a non-ROI.

FIG. 5A illustrates a dose of X-rays incident on an arbitrary straight line A-B across the ROI and the non-ROI. When the controller 160 moves the ROI filter 141 to a position corresponding to the non-ROI, as illustrated in FIG. 5A, a smaller dose of X-rays than that on the ROI is incident on the non-ROI. Since X-rays are also incident on the non-ROI, albeit in a small dose, information on an entire field of view can be obtained.

As described above, the X-ray imaging apparatus 100 may continuously perform X-ray imaging to obtain a video. As long as the ROI is in the object region, X-ray imaging may be performed while a difference between X-ray doses incident on the ROI and the non-ROI is maintained as illustrated in FIG. 5B.

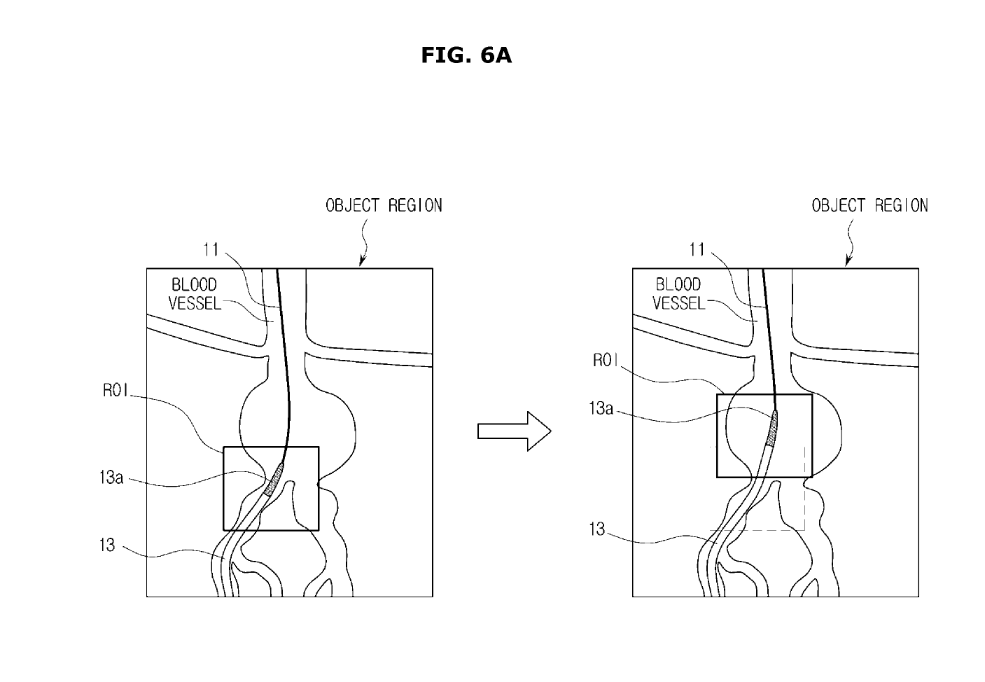

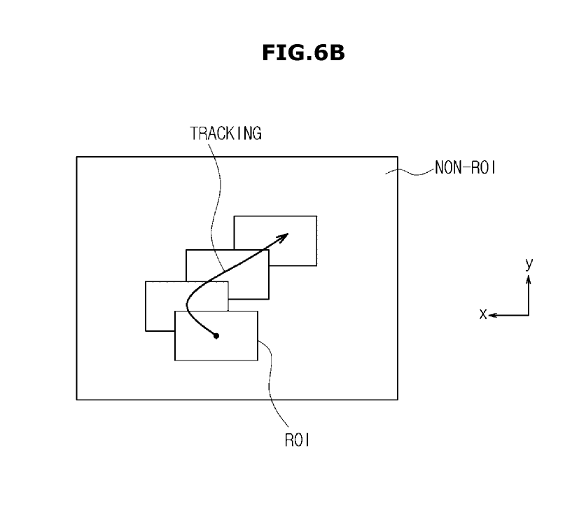

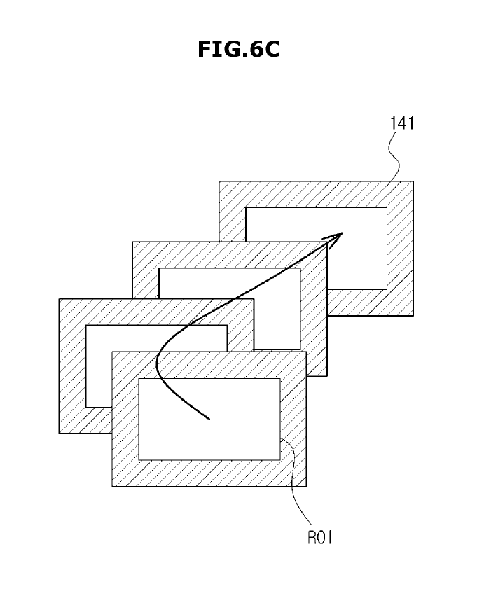

FIG. 6A is a diagram illustrating movement of an ROI according to movement of an object of interest. FIG. 6B is a diagram schematically illustrating an operation of tracking a moving ROI. FIG. 6C is a diagram illustrating movement of an ROI filter according to movement of an ROI.

As illustrated in FIGS. 6A, 6B and 6C, the X-ray video may show movement of an object in the object region. When the object in movement is the object of interest, the ROI may move according to the movement of the object of interest. As an example, as illustrated FIG. 6A, when a procedure of inserting a stent into a blood vessel is performed, the stent 13a, which is the object of interest, is moved to a target position in the blood vessel, and the ROI is also moved along with the movement of the stent 13a.

As described above, the image processor 150 may detect and track the object of interest in real time. When the ROI is moved, the image processor 150 tracks the movement in real time as illustrated in FIG. 6B, and the controller 160 moves the ROI filter 141 on the x-y plane such that the position of the ROI or the non-ROI is synchronized with the position of the ROI filter 141 as illustrated in FIG. 6C.

While moving the ROI and the ROI filter 141 according to the movement of the object of interest in the examples of FIGS. 6A, 6B, and 6C, a size of the ROI may be changed according to the movement of the object of interest. For example, when a size of the movement of the object of interest is not large, in other words, when a size of the movement is a predetermined threshold value or less, the image processor 150 may increase the size of the ROI according to the movement of the object of interest and fix the position of the ROI. In this case, since the ROI needs to include the moved object of interest while a position thereof is fixed, an increasing rate of the size of the ROI may be changed according to the movement of the object of interest.

The controller 160 may move the ROI filter 141 along the z axis such that the position of the ROI filter 141 along the z axis may be synchronized with a change in the size of the ROI. In an exemplary embodiment, a plurality of ROI filters may be provided in one disk, and when the disk rotates, the size of the ROI may be changed. An exemplary embodiment in which a plurality of ROI filters are provided in one disk and a size of the ROI is changed will be described below.

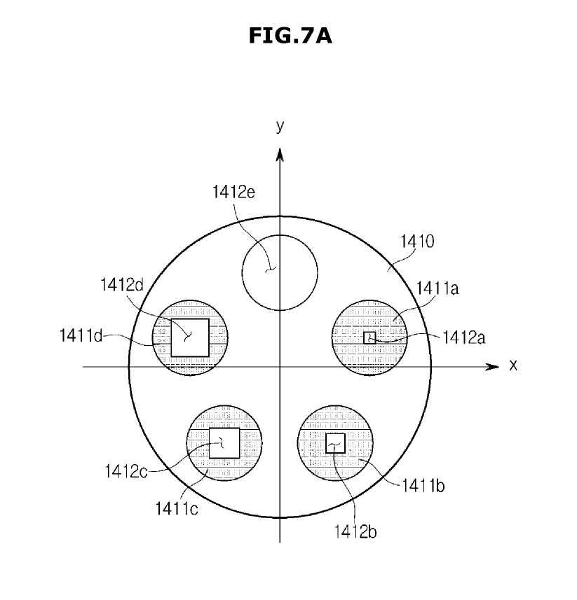

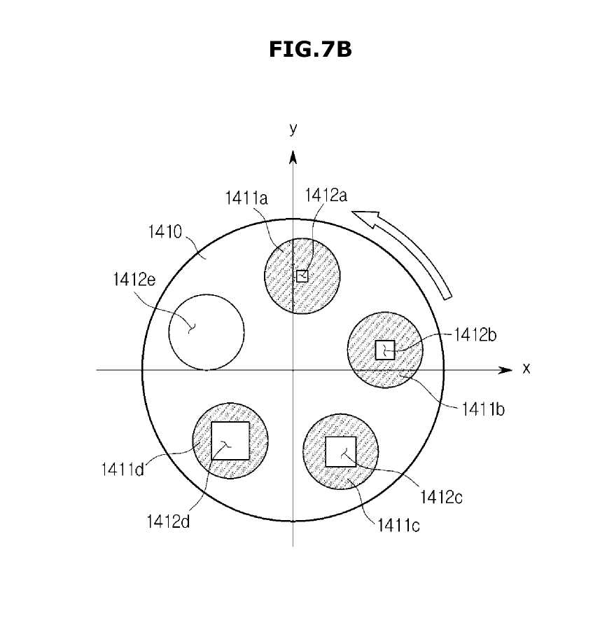

FIGS. 7A and 7B are diagrams illustrating a state in which a disk including ROI filters rotates according to a size of an ROI.

As illustrated in FIGS. 7A and 7B, the ROI filter 141 may include a plurality of ROI filters 1411a, 1411b, 1411c, and 1411d that are provided in one disk 1410. Openings 1412a, 1412b, 1412c, 1412d, and 1412e may be formed at centers of the ROI filters 1411a to 1411d. Openings 1412a to 1412d having different sizes may be formed in the plurality of ROI filters 1411a to 1411d. The openings 1412a to 1412d may have various shapes such as a polygon, a circle or an ellipse.

An exemplary embodiment in which the ROI filters 1411a to 1411d are provided in the circular disk 1410 and the rectangular openings 1412a to 1412d are formed in the ROI filters 1411a to 1411d will be described below with reference to FIGS. 7A and 7B. The shape of the disk 1410 and the shapes of the openings 1412a to 1412d are not limited to the above shapes, but various shapes may be used.

The ROI filters 1411a to 1411d are detachably mounted on the disk 1410. A plurality of openings 1412a to 1412d, having the same or similar shape as the opening 1412e, corresponding to the ROI filters 1411a to 1411d may be formed in the disk 1410. The ROI filters 1411a to 1411d may be mounted on the openings 1412a to 1412d. After the ROI filters 1411a to 1411d are mounted on the openings 1412a to 1412d, the openings 1412a to 1412d may have different sizes as shown in FIGS. 7A and 7B.

The plurality of openings 1412a to 1412e may be disposed on the disk 1410 in a circumferential direction with a predetermined interval therebetween. The openings 1412a to 1412d may be disposed such that sizes of the openings 1412a to 1412d after the ROI filters 1411a to 1411d are mounted thereon increase in a clockwise direction. The openings 1412a to 1412d are positioned in the ROI of the object, and doses of X-rays passing through the openings 1412a to 1412d are maintained. X-rays passing through the ROI filters 1411a to 1411d are filtered, and smaller X-ray doses may pass therethrough.

No ROI filter may be mounted on at least one opening 1412e. The opening 1412e in which no ROI filter is disposed may be positioned between the ROI filter 1411a in which the opening 1412a having a smallest size is formed and the ROI filter 1411d in which the opening 1412d having a largest size is formed among the ROI filters 1411a to 1411d disposed in the disk 1410. When the opening 1412e is positioned in the ROI of the object, X-rays generated from the X-ray source 110 may pass through the opening 1412e without being filtered and a dose thereof may be maintained.

Referring to FIG. 7B, the controller 160 may drive the filter driver 143, rotate the disk 1410 in a clockwise direction or a counter-clockwise direction, and change an area to which non-filtered X-rays are radiated. For example, the disk 1410 rotates in the counter-clockwise direction 710, the ROI filter 1411a including the opening 1412a having the smallest size is moved to be positioned on the y axis, and the ROI filter 1411b including the opening 1412b having a second smallest size may be moved to be positioned on the x axis. Therefore, an area to which non-filtered X-rays are radiated on the ROI positioned on the x axis may increase.

In this manner, when the disk 1410 on which the plurality of ROI filters 1411a to 1411d having the openings 1412a to 1412d of different sizes are mounted rotates, an area to which non-filtered X-rays are radiated on the ROI may be controlled without moving the ROI filter in a z axis direction.

When the ROI filter is provided, it is possible to decrease a dose of X-rays radiated to the non-ROI within the object when imaging is performed by the X-ray imaging apparatus 100. Without moving the ROI filter along the z axis, an area to which non-filtered X-rays are radiated to the ROI may be controlled according to rotation of the disk. Since the ROI filter does not need to move along the z axis, it is possible to decrease a volume of the filter 140.

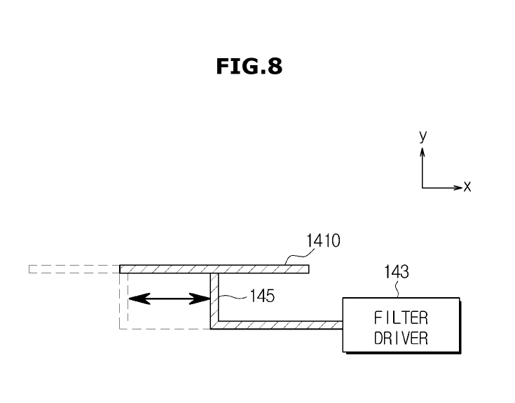

FIG. 8 is a side cross-sectional view of a disk including an ROI filter according to an exemplary embodiment.

As illustrated in FIG. 8, the disk 1410 having the ROI filter 1411 may move on the x-y plane by a drive shaft 145 connected to the filter driver 143. According to the movement of the object of interest, a position and a size of the ROI may be changed. As illustrated in FIGS. 7A and 7B, the size of the ROI may be controlled by rotation of the disk 1410, and the position of the ROI may be controlled when the disk 1410 moves on the x-y plane due to the drive shaft 145. Therefore, a dose of X-rays radiated to the object may decrease, and an X-ray image of the moving object of interest may be obtained.

An area of the ROI and the non-ROI to which X-rays are radiated and an X-ray dose on the ROI and the non-ROI according to a size of the opening formed in the ROI filter will be described below.

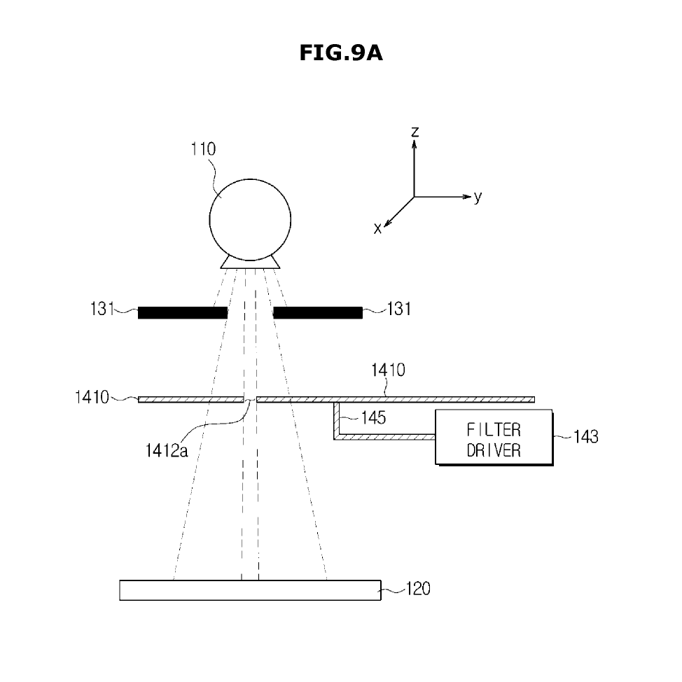

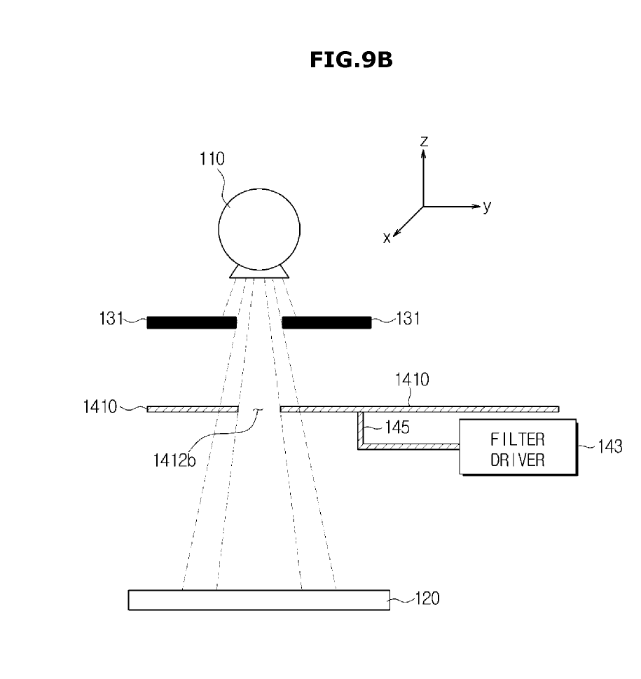

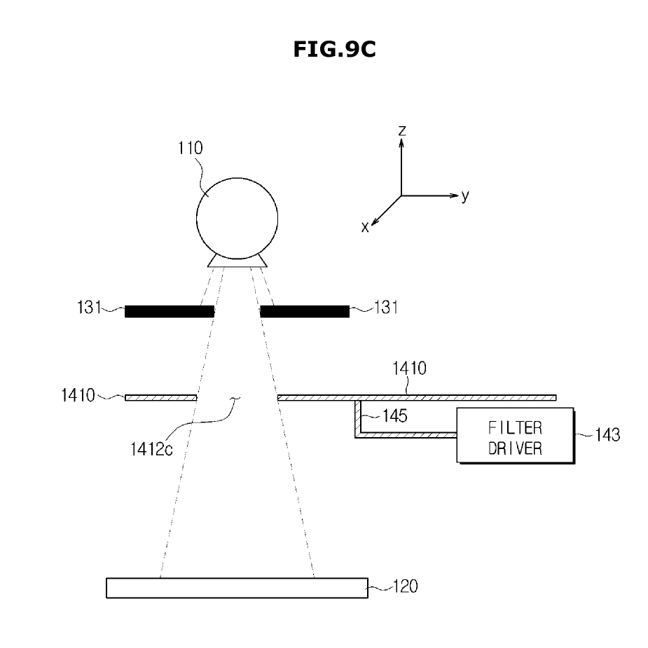

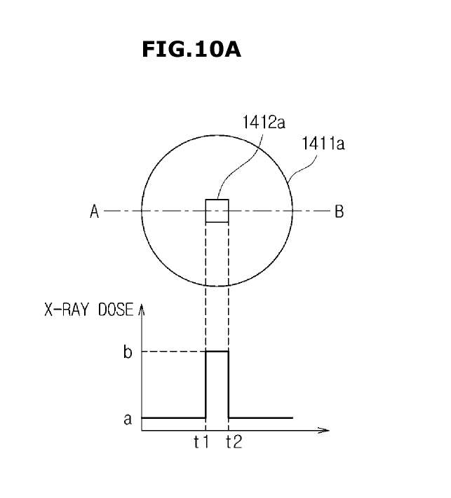

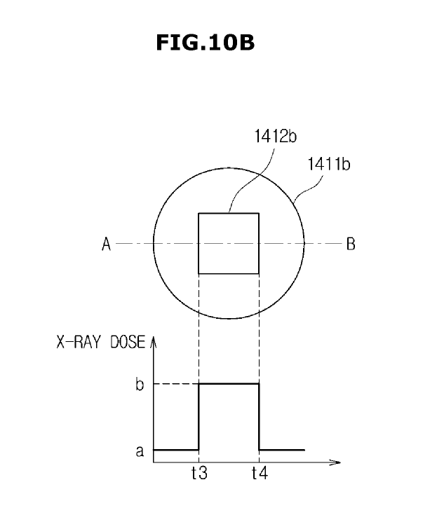

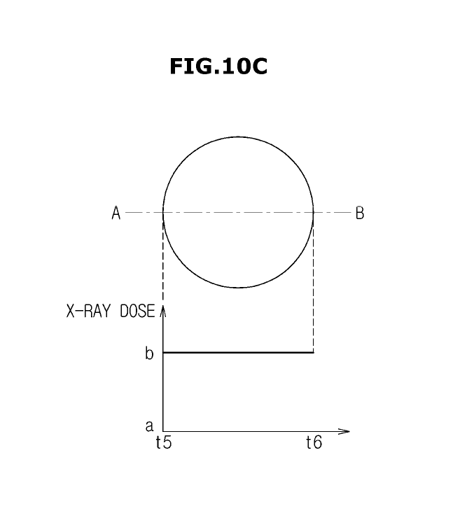

FIGS. 9A to 9C are side cross-sectional views of an X-ray imaging apparatus including a disk having an ROI filter. FIGS. 10A to 10C are diagrams schematically illustrating doses of X-rays incident on an ROI and a non-ROI.

As illustrated in FIGS. 9A and 10A, a dose of X-rays that are generated from the X-ray source 110 and pass through a first opening 1412a having the smallest size of a first ROI filter 1411a may be maintained (b: refer to FIG. 10A). The first opening 1412a may be positioned corresponding to the ROI within the object region. X-rays passing through the first ROI filter 1411a positioned in the vicinity of the first opening 1412a has a smaller dose and may be radiated to the non-ROI (a: refer to FIG. 10A). X-rays passing through the first opening 1412a are not filtered corresponding to the ROI part, and a dose (b) of X-rays generated from the X-ray source 110 may be radiated without filtering through the first opening 1412a corresponding to an interval t1 to t2.

When the size of the ROI increases, the disk 1410 may rotate, and a second opening 1412b of a second ROI filter 1411b may be positioned in the ROI. The second opening 1412b may have a size larger than the first opening 1412a. X-rays that are generated from the X-ray source 110 and pass through the second opening 1412b may be radiated to the ROI without filtering. The ROI may include an area corresponding to an interval t3 to t4, which is defined by the second opening 1412b. A dose (b) of X-rays passing through the second opening 1412b may be substantially the same as a dose of X-rays passing through the first opening 1412a. A smaller dose (a) of X-rays pass through the second ROI filter 1411b positioned in the vicinity of the second opening 1412b and may be radiated to the non-ROI.

As the size of the ROI becomes larger, the disk 1410 further rotates, and thus the opening 1412e on which the ROI filter 1411 is not mounted may be positioned corresponding to the ROI. X-rays that are generated from the X-ray source 110 and pass through the opening 1412e may be radiated to the ROI without filtering. The ROI may include an area corresponding to an interval t5 to 56, which is defined by the opening 1412e. A dose of X-rays generated from the X-ray source 110 may pass through the opening 1412e without filtering and radiated to the ROI.

In this manner, when the size of the ROI is changed, the disk 1410 may quickly rotate and the ROI filter is changed. Therefore, a sufficient dose of X-rays may be radiated to the ROI. Also, when a smaller dose of X-rays is radiated to the non-ROI, a dose of X-rays radiated to the object may be reduced when X-ray imaging is performed.

While an exemplary embodiment in which the openings 1412a to 1412e are provided in a rectangular shape has been described above, the opening formed in the ROI filter 1411 may be formed in a circular shape or various other shapes.

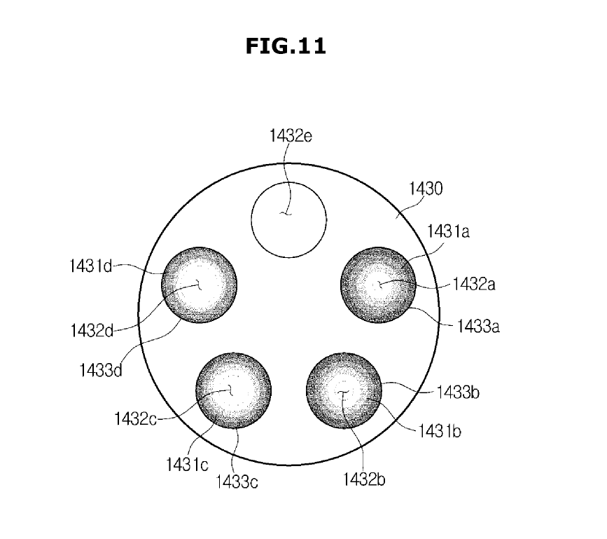

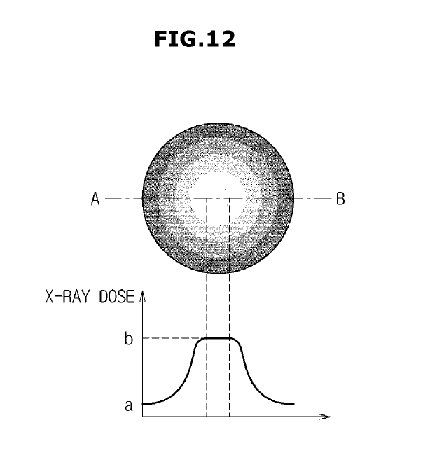

FIG. 11 is a diagram illustrating a disk including ROI filters according to another exemplary embodiment. FIG. 12 is a diagram schematically illustrating doses of X-rays incident on an ROI and a non-ROI according to another exemplary embodiment.

As illustrated in FIGS. 11 and 12, a plurality of ROI filters 1431a, 1431b, 1431c and 1431d may be provided such that doses of X-rays passing through the ROI filters 1431a to 1431d gradually decrease in a direction from a center toward an outer diameter of the ROI filters 1431a to 1431d. The ROI filters 1431a to 1431d may be respectively mounted on openings 1432a, 1432b, 1432c, and 1432d formed on a disk 1430. The plurality of openings 1431a to 1432e are formed on the disk 1430. The plurality of ROI filters 1431a to 1431d may be mounted on the disk 1430 including the plurality of openings 1431a to 1432e.

The openings 1432a to 1432d may be positioned at the centers of the ROI filters 1431a to 1431d. The openings 1432a to 1432d of different sizes may be formed in the plurality of ROI filters 1431a to 1431d mounted on the disk 1430. As an example, in the circular disk 1430, the ROI filters 1431a to 1431d may be positioned such that a first ROI filter 1431a in which a first opening 1432a having a smallest size is formed to a fourth ROI filter 1431d in which a fourth opening 1432d having a largest size is formed in a clockwise direction.

The openings 1432a to 1432d may be positioned corresponding to the ROI of the object of interest. X-rays generated from the X-ray source 110 may pass through the openings 1432a to 1432d while doses of the X-rays are maintained and may be radiated to the ROI. X-rays generated from the X-ray source 110 may pass through the ROI filters 1431a to 1431d and smaller doses of the X-rays may be radiated to the non-ROI than doses of X-rays passing through the openings 1432.

In the ROI filters 1431, parts of the ROI filters 1431a to 1431d positioned in outer diameters 1433a to 1433d may have greater thicknesses than parts of the ROI filters 1431a to 1431d adjacent to the openings 1432. The ROI filters 1431a to 1431d may have thicknesses that gradually increase from the openings 1432a to 1432d to the outer diameters 1433. Doses of X-rays passing through the ROI filters 1431a to 1431d adjacent to the outer diameters 1433a to 1433d may be smaller than doses of X-rays passing through the ROI filters 1431a to 1431d positioned adjacent to the openings 1432.

When the ROI filters 1431a to 1431d have thicknesses that gradually increase from the openings 1432a to 1432d to the outer diameters 1433, doses of X-rays passing through the ROI filters 1431a to 1431d and the openings 1432a to 1432d may substantially form a normal distribution as illustrated in FIG. 12. X-rays generated from the X-ray source 110 may be radiated to the ROI through the openings 1432a to 1432d while doses (b) thereof are maintained. Doses of X-rays passing through the ROI filters 1431a to 1431d may gradually decrease from the openings 1432a to 1432d to the outer diameters 1433a to 1433d of the ROI filters 1431. A dose (a) smaller than the doses (b) of X-rays passing through the openings 1432a to 1432d may be radiated to the non-ROI corresponding to the outer diameters 1433a to 1433d.

There may be no ROI filter that is mounted in at least one of the plurality of openings 1432e formed in the disk 1430 if needed. X-rays passing through the opening 1432e may be radiated to the ROI while a dose of X-rays generated from the X-ray source 110 is maintained.

While the exemplary embodiment in which the openings 1432a to 1432d are provided at the centers of the ROI filters 1431a to 1431d has been described above, no opening may be provided to the ROI filters 1431a to 1431d and a dose of X-rays passing through the ROI filters 1431a to 1431d may decrease toward an outer diameter from the center of the ROI filter 1431.

Similar to the example illustrated in FIG. 8, the drive shaft 145 connected to the filter driver 143 may be mounted on the disk 1430 on which the ROI filters 1431a to 1431d are mounted. The disk 1430 may be provided on the x-y plane to be capable of being moved by the drive shaft 145. The disk 1430 may move on the x-y plane according to the movement of the ROI. The disk 1430 rapidly rotates according to an area of the ROI, and the ROI filters 1431a to 1431d in which the openings 1432a to 1432d having sizes appropriate for imaging an area of the ROI are formed or the opening 1432e formed in the disk 1430 may be positioned corresponding to the ROI.

The ROI filters 1431a to 1431d which control doses of X-rays passing therethrough to decrease from the center to the outer diameter of the ROI filters 1431a to 1431d as illustrated in FIGS. 11 and 12 and the ROI filters 1411 in which the openings 1412a to 1412e are formed as illustrated in FIGS. 7A and 7B may be mounted on the same disk.

In this manner, the plurality of ROI filters may be provided in one disk, the disk rotates according to an area of the ROI, and thus X-rays generated from the X-ray source may be appropriately radiated to at least a portion or the entire ROI while a dose thereof is maintained. Since the ROI filter need not be moved in a z axis direction to control an area to which non-filtered X-rays are radiated, it is possible to substantially decrease a volume of a filter and decrease a volume of the X-ray imaging apparatus.

By providing a smaller dose of X-rays than that on an ROI to be incident on a non-ROI using an ROI filter, it is possible to implement low dose X-ray imaging and reduce a loss of a field of view of an X-ray image.

At least one of the components, elements or units represented by a block as illustrated in the block diagram of FIG. 1 may be embodied as various numbers of hardware, software and/or firmware structures that execute respective functions described above, according to an exemplary embodiment. For example, at least one of these components, elements or units may use a direct circuit structure, such as a memory, processing, logic, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions. Also, at least one of these components, elements or units may further include a processor such as a central processing unit (CPU) that performs the respective functions, a microprocessor, or the like. Functional aspects of the above exemplary embodiments may be implemented in algorithms that are executed by one or more processors and stored in a computer readable recording medium. Furthermore, the components, elements or units represented by a block or processing steps may employ any number of related art techniques for electronics configuration, signal processing and/or control, data processing and the like.

Although a few embodiments have been shown and described, it would be appreciated by those skilled in the art that changes may be made in the exemplary embodiments without departing from the principles and spirit of the disclosure, the scope of which is defined in the claims and their equivalents.

What is claimed is:

1. An X-ray imaging apparatus comprising:
an X-ray source configured to radiate X-rays to an object;
an X-ray detector configured to detect X-rays radiated by the X-ray source; and
a disk rotatably provided between the X-ray source and the X-ray detector, wherein region of interest (ROI) filters are configured to filter X-rays radiated by the X-ray source are mounted on the disk, the ROI filters having openings of different sizes.

2. The X-ray imaging apparatus according to claim 1, wherein the ROI filters are configured to filter the X-rays such that the filtered X-rays have a smaller dose than the X-rays passing through the openings of the ROI filters.

3. The X-ray imaging apparatus according to claim 1, further comprising:
a controller configured to control such that the disk is moved or rotated according to movement of a region of interest (ROI) of the object or a size of the ROI.

4. The X-ray imaging apparatus according to claim 3, wherein the controller is configured to control such that the disk rotates about an axis that is parallel to a line connecting the X-ray source and the X-ray detector according to the size of the ROI.

5. The X-ray imaging apparatus according to claim 3, wherein the controller is configured to control such that the disk moves on a plane that is perpendicular to a line connecting the X-ray source and the X-ray detector according to the movement of the ROI.

6. The X-ray imaging apparatus according to claim 1, wherein a number of the ROI filters having the openings is less than a number of openings formed on the disk.

7. The X-ray imaging apparatus according to claim 1, wherein the ROI filters are detachably mounted on the disk.

8. The X-ray imaging apparatus according to claim 1, wherein a thickness of an ROI filter in an area adjacent to an opening is smaller than a thickness of the ROI filter in an area adjacent to an outer diameter of the ROI filter.

9. The X-ray imaging apparatus according to claim 3, wherein the controller is configured to control to rotate the disk such that the X-rays are filtered by an ROI filter having an opening of which a size corresponds to the size of the ROI.

10. The X-ray imaging apparatus according to claim 1, wherein an ROI filter has a circular shape.

11. The X-ray imaging apparatus according to claim 1, wherein an opening has a circular shape.

12. The X-ray imaging apparatus according to claim 1, wherein an opening has a polygonal shape.

13. The X-ray imaging apparatus according to claim 1, wherein the X-ray detector is configured to detect the X-rays radiated to the object and obtains a plurality of frame images by using the detected X-rays.

14. The X-ray imaging apparatus according to claim 13, further comprising:
an image processor configured to obtain information on the ROI from at least one of the plurality of frame images and transmit the information to a controller, wherein the controller controls the disk based on the information.

15. The X-ray imaging apparatus according to claim 14, wherein the image processor is configured to detect an object of interest from the frame image and set the ROI based on at least one of a position, a size, and a movement of the object of interest.

16. A method of controlling an X-ray imaging apparatus comprising a disk on which a plurality of ROI filters having openings of different sizes are mounted, the method comprising:
radiating, by using an X-ray source, X-rays to an object;
detecting, by using an X-ray detector, the radiated X-rays and obtaining information on a region of interest (ROI) of the object; and
controlling the disk positioned between the X-ray source and the X-ray detector to rotate corresponding to an area of the ROI.

17. The method according to claim 16, wherein the controlling comprises:
controlling the disk to move on a plane that is parallel with a plane on which the ROI is positioned.

18. The method according to claim 17, wherein the controlling further comprises controlling the disk to rotate about an axis that is parallel to a line connecting the X-ray source and the X-ray detector and move on a plane that is perpendicular to the line connecting the X-ray source and the X-ray detector.

19. An X-ray imaging apparatus, comprising:
a plurality of region of interest (ROI) filters having openings of different sizes and configured to filter X-rays generated from an X-ray source, the filtered X-rays being radiated to an object;
a disk on which the plurality of ROI filters are mounted; and
a controller configured to control such that the disk is rotated or moved corresponding to a region of interest (ROI) of the object.

20. The X-ray imaging apparatus according to claim 19, wherein the controller is configured to move the disk on a plane that is parallel with a plane on which the ROI is positioned.
